(19) United States
(12) Patent Application Publication
(10) Pub. No.: US 2017/0246028 A1
(43) Pub. Date: Aug. 31, 2017

(54) Body Cooling Device

(71) Applicants: Colin Paul Cahoon, Dallas, TX (US); Susan Lyon Cahoon, Dallas, TX (US)

(72) Inventors: Colin Paul Cahoon, Dallas, TX (US); Susan Lyon Cahoon, Dallas, TX (US)

(21) Appl. No.: 15/053,837

(22) Filed: Feb. 25, 2016

(51) Int. Cl. A61F 7/00

(52) U.S. Cl. CPC A61F 7/0085; A61F 2007/001; A61F 2007/0019; A61F 2007/0064

(57) ABSTRACT

A portable body cooling device, worn on the upper torso of a person, that uses a compressed or liquefied gas cartridge to vent cool gas onto the skin of the wearer as well as to act as a heat sink during the gas release. The cartridge is in contact with a conductive material that transfers heat from the skin of the wearer to the cartridge. The cooling device provides immediate and temporary relief from heat flashes.

### BACKGROUND OF THE INVENTION

Technical Field

The present invention relates to a portable device for cooling the human body. The device relies on the sudden release of compressed or liquefied gas to cool the surface or skin of the human body, and, specifically, operates by the metered release of compressed gas from a small container, that is carried on the body or by the user, into gas distribution channels to direct the released gas onto the body of a person wearing the device. The device also conducts heat away from the body when activated, by use of metal or other heat conducting components in communication with the gas container and the body. The device is disguised as a jewelry piece, is built into a woman's bra, or is attached to a woman's bra in various preferred embodiments.

Background of the Invention

Hot flashes are a condition typically involving the experience of a feeling of intense heating with sweating and rapid heartbeat lasting typically for a few minutes. The condition is a common symptom of menopause and perimenopause. The sensation of heat begins and ends rapidly and is usually most intense in the face and chest area, but can also be experienced in the neck and upper back. These episodes can start unpredictably at any time and may occur as frequently as every few minutes. Women who are prone to hot flashes feel as if their upper torso is suddenly overheating and in need of immediate cooling. A typical hot flash comes on rapidly and reaches a maximum intensity in as little as a minute. Once the episode passes, however, the feeling of normalcy returns.

Hot flashes can also be experienced by young women as, for example, a symptom of a problem with the pituitary gland. Men with low testosterone, prostate cancer, testicular cancer, and andropause can also experience hot flashes. The condition, though, is quite common in middle-age women going through menopause.

Symptoms of menopause, such as hot flashes, can sometimes be treated with hormone replacement therapy. Such therapy, however, can have significant long and short-term risks such as breast cancer, dementia, and stroke. The most commonly followed treatment is to remove clothing and seek cool air immediately upon the onset of a hot flash episode. Opening one's blouse to the inside of a freezer can bring instant relief, but is not a practical solution in most settings. Even using a hand-held folding fan can provide some limited relief during a hot flash, but the use of ambient air is minimally effective and the fanning brings attention to the user.

A need exists, therefore, for a new and novel device and method for the treatment of hot flash symptoms. The device should be portable, discreet, and provide for immediate body cooling over a time period that corresponds to the duration of a hot flash episode. Ideally, such innovation should be relatively inexpensive and provide a flexible platform across many different clothing options and body shapes.

### SUMMARY OF THE INVENTION

In a preferred embodiment, the invention comprises a personal cooling device which incorporates a gas container for storing a pressurized or liquefied gas, such as CO2 cartridges commonly used in home soda machines, for use as portable bike tire pumps, or in air guns and rifles. Such cartridges are typically made of metal and can be threaded at a discharge end or non-threaded. When the gas is released from such container it expands rapidly, absorbing ambient heat, thereby causing the exiting gas to chill and cooling the gas container as well. The invention described herein takes advantage of this thermodynamic cooling to help cool the exterior of a person who is experiencing a hot flash.

The gas “container” or “cartridge,” such terms are used interchangeably herein, is fitted with a valve that can be operated by a wearer of the cooling devices and is integral to at least a portion of the cooling device. The valve is further in communication with one or more gas distribution channels, each having one or more gas ports. When the valve is activated, the chilled gas is ported onto the skin of the wearer, typically in the area of the upper torso (chest, neck, and upper back). The exhausting of the chilled gas onto the skin of the wearer has the effect of blowing cold or cool air onto the person similar to the effect of standing in front of an open freezer door or air-conditioning vent.

Further, in a preferred embodiment, the portion of the device in which the cartridge resides is constructed of a heat conducting (conductive) material, such as metal or cloth with metal threads, and is in contact with the skin of the wearer of the device. As a consequence, when the valve is activated the cartridge rapidly cools and draws heat away from the skin of the wearer in the area that is in contact with the heat conducting material. This dual cooling functionality from both exhausted chilled gas and heat transfer due to conductivity from the warm skin to the cold gas container significantly lowers the exterior temperature of the wearer for a short period of time. Shortly after the valve is closed the device returns to ambient temperature to coincide with the short duration of the hot flash.

The cooling device is available in several different embodiments and configurations. For example, in one embodiment the cooling device is an insert that attaches to the center of the front of a woman's bra. The cartridge in this embodiment is located under the bra in between the breasts (or on top of the center span of the bra) or can be configured such that one cartridge is located under each breast in a “push-up” arrangement. A plurality of gas ports, in communication with the one or two cartridges, direct vented gas upwards towards the neck area. A conductive frame that holds the cartridge conducts heat from around the chest to the cartridge.

In an alternative embodiment the device is connected to a piece of tubing that is attached at one side of the frame, proceeds up the chest area, over the shoulder, around the back of the neck, and back down to the other side of the frame, much like a necklace would attach to a pendant. This tube, in one embodiment, is hidden under clothing. In another embodiment the tube is visible and disguised as a necklace. The tube also contains gas ports and is in communication with the valve and cartridge. Consequently, when the valve is activated chilled gas is ported to the skin area of the wearer along the chest, neck, and upper back areas. The cartridge can be attached to clothing, suspended from the tubing, or located in a carrier worn by the user.

In another embodiment the cooling device is built into a woman's bra. In this embodiment the cartridge resides in a metal frame in the center of the front of the bra or two cartridges are used with one each located below a breast in a “push up” arrangement, much as the embodiment discussed above. Tubing is incorporated in the bra itself, such as along the top of the front of the bra and, alternatively, along the bra straps over the shoulders or around the neck, such that chilled gas is ported out of the tubes to the chest, neck, and upper back areas. Heat conducting material, such as metal mesh or metallic fibers, is incorporated in the bra itself in order to direct heat from the chest to the cartridge.

In another embodiment the cooling device comprises a pendant hung around the wearer's neck. In this embodiment the cartridge resides in the pendant. Gas is exhausted to the chest and neck area through ports in both the pendant and the necklace that suspends the pendant, or just through the top of the pendant. The necklace is preferably made of a heat conducting material such as metal to help transfer heat from the skin to the pendant where the cartridge resides.

What is provided is a unique and novel invention that offers immediate but temporary relief of the symptoms of a hot flash. The device in its various embodiments is portable, discreet, and provides for immediate body cooling over a time period to correspond to the duration of a hot flash episode. The innovation provided is relatively inexpensive and provides a flexible platform across many different wearing applications.

### BRIEF DESCRIPTION OF THE DRAWINGS

The novel features believed characteristic of the invention are set forth in the appended claims. The invention itself, as well as a preferred mode of use, further objectives and advantages thereof, will be best understood by reference to the following detailed description of illustrative embodiments when read in conjunction with the accompanying drawings, wherein:

FIG. 1 is a schematic illustration of the basic components of one embodiment of the invention;

FIG. 2a through 2c illustrate embodiments of the invention; and

FIG. 3 illustrates one embodiment of the invention incorporated with a bra.

### DETAILED DESCRIPTION

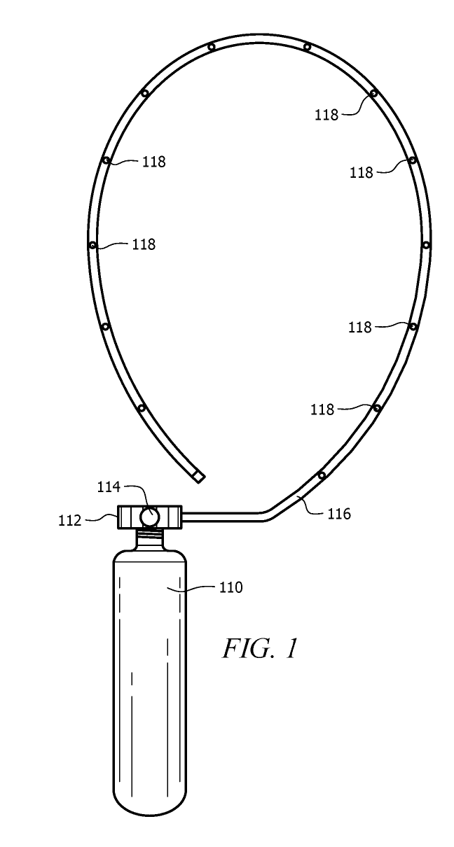

Referring to FIG. 1, shown are the basic components of one embodiment of the cooling device. A gas container such as a CO2 cartridge 110 is shown attached to and in gas communication with a valve 112. The valve 112 is attached to the cartridge 110 by threading on the discharge end of the cartridge 110 in an embodiment that utilizes cartridges 110 manufactured with threading. In such embodiment the top of the cartridge 110 acts as a male insert that is screwed into a threaded female opening on the valve 112. Any embodiment using a cartridge 110 that does not have threading at the top of the cartridge requires that the valve be integral with a cartridge enclosure. The enclosure (not shown) holds the cartridge 110 in place while the valve 112 is forced down over the end of the cartridge, thereby puncturing the end of the cartridge 110 and pressurizing the valve 112. Similar enclosures can be found in the prior art for use as portable bicycle tire inflation devices, for example.

Also shown in FIG. 1 is a tube 116 (or gas discharge channel) connected to and in gas communication with the valve 112. The tube 116 has a plurality of gas ports 118. The valve 112 has a button or other triggering mechanism 114 that activates or opens the valve 112 in order to provide a metered gas release into the tubing 116. The released gas then exits the tubing 116 through the gas ports 118. The tube 116, in one embodiment, is worn around the neck of a user like a necklace and is sealed at one end 17. The tube is most effective when constructed of a heat conducting material such as metal or metal fibers, because the conductive material conducts heat away from the body of the user and to the cartridge 110. The cartridge 110 becomes quite cold when gas is released therefrom and acts as a heat sink for the excess body heat given off during a hot flash episode.

The valve 114 in all embodiments discussed is either mechanically activated by direct contact from the user or remotely activated by, for example, a control device that transmits to a receiver that activates the valve 114. Such control device can be stored, for example, in a purse or pocket of the user.

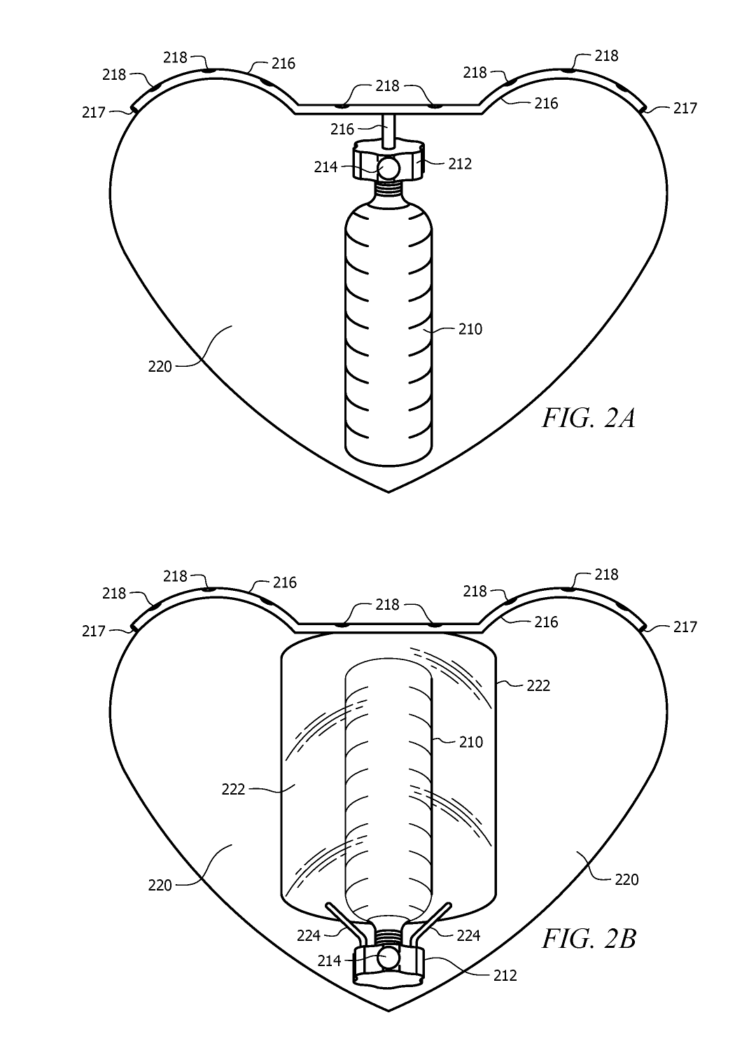

With the basic components of the device in mind, FIGS. 2a through 2c provide more detail on two embodiments of the device. FIG. 2a shows a gas cartridge 210 mounted on or connected to a frame 220. This frame 220 is constructed at least partially of a conductive material such as metal, metal mesh, or metal thread. In one embodiment a soft and pliable metal is used, such as copper or aluminum. In other embodiments the metal is a thin metal sheet, a chain maille, a metal mesh, metal fibers either weaved together or in combination with other materials, or combinations thereof depending on the embodiment. The conductive material is in physical contact with the cartridge 210 and the body/skin of the wearer in order to conduct heat away from the body to the cartridge 210. The frame 220 further comprises an insulation or insulative material on surfaces of the frame 220 that are not in contact with the cartridge 210 or the body/skin of the wearer in order to limit the amount of heat transfer from the ambient surroundings. Such insulation material includes plastics, foam, cloth, ceramics, wood, and combinations thereof. Thus, the frame 220 comprises heat conductive material on a first side (in contact with the wearer) and insulative material on a second side (exposed to ambient).

The cartridge 210 is again connected to and in gas communication with a valve 212 having a release button or mechanism 214. The valve 212 is in turn connected to and in gas communication with one or more gas distribution channels 216, such as tubing. In the embodiment illustrated in FIG. 2a, the gas distribution channel 216 starts as a single tube 216 and then splits in two directions across the top of the frame 220. The gas distribution channel 216 has a plurality of gas ports 218 oriented to vent gas onto the skin of the user and is sealed at each end 217.

The frame 220 is shaped to fit the contours of the chest area of the wearer. In the embodiment illustrated in FIG. 2a, the frame is placed in the center of a woman's chest such that the cartridge is centered between the woman's breasts. The frame 220 can be attached to a bra or other article of clothing, such as a vest, blouse, or shirt. The frame 220 is then in physical contact with area between the wearer's breasts and an upper portion of each breast with the gas ports 218 oriented to vent gas onto the upper chest area towards the wearer's neck. When the trigger mechanism 214 is activated, the valve opens and releases pressurized CO2 from the cartridge 210. The cartridge 210 begins a rapid cooling which draws heat away from the wearer's body from the areas in contact with the conductive portions of the frame 220.

In an alternative embodiment the basic components of the device illustrated in FIG. 2a can be worn as a pendant around a woman's neck. The pendant rests on the wearer's chest. The gas distribution channel 216 is located at the top of the pendant and, in one embodiment, is contiguous with a necklace that supports the pendant. The necklace, in one embodiment, consists of a conductive gas distribution channel with a plurality of gas ports such that vented gas is directed around the wearer's neck, upper back, and chest area.

FIG. 2b illustrates another embodiment of the device similar to the embodiment illustrated in FIG. 2a. Again, a cartridge 210 is shown mounted with a frame 220. FIG. 2c is a schematic representation of this embodiment looking at the base of the device in a cutaway view. The view in FIG. 2c is obtained by rotating the plan view of FIG. 2b 90 degrees about a horizontal axis such that the top of the valve 212 is shown atop the cartridge 210.

As shown in FIG. 2c, the cartridge 210 is encased in an airtight compartment 250 defined by a portion of the frame 220 and a cover 222. This airtight compartment 250 can also be described as a gas manifold. This compartment 250 comprises heat conductive material in at least the inner surfaces of the compartment 250. The exterior surface of the cover 222 comprises an insulation material in order to minimize the heat transfer from areas other than the interior of the compartment 250.

Referring to both FIGS. 2b and 2c, when the trigger mechanism 214 is activated, the valve 212 releases cool gas into the compartment 250 via tubing 224 or other gas channeling means. The released gas then flows through the compartment or gas manifold 250, past the exterior of the cartridge, and into the gas distribution channel 216 located at the top of the device. In this embodiment the gas manifold 250 is in between and in communication with the valve 212 and at least one gas distribution channel 216. The cartridge 210 simultaneously and rapidly cools during the gas release due to the sudden expansion of the gas as it exits the cartridge 210. Heat in the exhausting gas is further removed as it travels through the compartment 250 and past the cartridge 210. Consequently, the compartment 250 and encased cartridge 210 act as a heat exchanger when the valve 212 is activated.

It can be seen in FIG. 2b that the top of the cartridge 210 is oriented at the bottom of the frame 220. This is done to facilitate the heat exchanging flow of the gas through the compartment 250. Thus, the portable cooling device shown comprises a frame 220 that further comprises a gas manifold 250 through which gas released from the gas container 210 travels prior to entering at least one gas distribution channel 216.

FIG. 2c also shows a gross representation of the shape of the frame 220 as discussed in reference to the embodiment illustrated by FIG. 2a. The frame 220 of the cooling device is shaped such as to fit the contours of the wearer when located between the wearer's breasts. Thus, the frame 220 is shaped to fit the contours of a wearer's breasts. As with the embodiment illustrated in FIG. 2a, the embodiment illustrated in FIGS. 2b and 2c has gas ports 218 at the top of the frame 220 which direct vented gas onto the upper chest area of the wearer toward the neck. Again, the gas distribution channel 216 is sealed at each end 217, but can be augmented by tubing around the neck of the wearer.

In an alternative embodiment to either of the embodiments illustrated in FIGS. 2a through 2c, the gas distribution channel 216 can comprise conductive tubing that runs along the exterior of the cartridge 210 before the area of the channel 216 comprising gas ports 218. This alternative embodiment facilitates the heat exchanging functionality without the need for an airtight compartment 250. In this embodiment the gas manifold is the portion of the gas distribution channel 216 in contact with the cartridge 210.

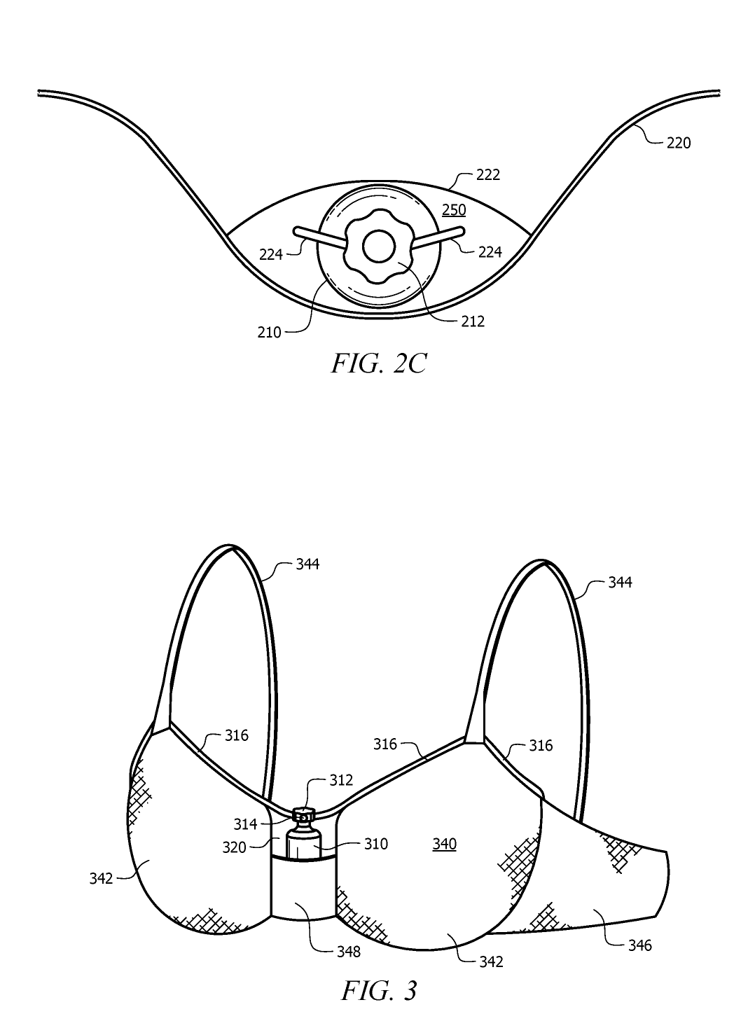

FIG. 3 illustrates an embodiment of the invention incorporated in a women's bra 340. This embodiment consists of either a kit that is installed on the bra 340 or a bra 340 that has the invention built into the bra 340 prior to retail sale. Shown is a generic bra 340 construction with two cups 342, a center span 348 connecting the two cups 342, a horizontal strap 346 (a second horizontal strap is not visible) and shoulder straps 344. It should be understood that, while FIG. 3 illustrates an embodiment of a bra having shoulder straps 344, the principles of the invention described in relation to said shoulder straps 344 are contemplated to apply equally to a bra having a halter top arrangement with a strap around the wearer's neck.

In one embodiment the cartridge 310 is mounted to or in contact with a frame 320. Depending on the embodiment, the cartridge 310 resides either hooked over the center span 348 while in communication with frame 320, or the cartridge 310 resides under the center span 348. The frame comprises a heat conductive material such has a metal, or metal fiber. The metal is presented as a thin pliable sheet, a chain maille, a metal mesh, metal fibers either weaved together or in combination with other materials, or combinations thereof depending on the embodiment. The frame 320 is shown as residing between the two cups 342 above the center span 348, but it should be understood that, depending on the embodiment, the frame can extend into part or all of the cups 342 and down into all or part of the center span 348. Such portion of the frame 320 is between the skin of the wearer/user and the rest of the bra material in order to facilitate heat transfer from the wearer/user to the cartridge 310. Portions of the frame 320 not in contact with the wearer's skin are covered with an insulation material such as cloth, plastic, or foam.

As with previously discussed embodiments, the cartridge is connected to and in gas communication with a valve 312 that is activated by a trigger mechanism 314. The valve is further connected to and in gas communication with tubing 316 that is shown running along the top of each cup 342. In an alternative embodiment the tubing 316 also runs along all or part of the horizontal strap 346 and/or all or part of the shoulder straps 344. The tubing (or other type of gas distribution channel) 316 has a plurality of gas ports (not shown) which direct vented gas onto the upper torso of the wearer.

Another embodiment of the bra concept of the invention uses two cartridges 310, one each placed in a roughly horizontal arrangement at the bottom of each cup 342 between the bra and the wearer's breasts. In this embodiment the cartridges 310 are in a “pushup” arrangement below the wearer's breasts, thus enhancing the bust line of the wearer as is common with pushup bras presently in the market.

Another embodiment of the cooling device locates two gas cartridges 310 on the outside of each cup 342 near the side straps 346. In such embodiment the cartridges 310 are oriented in a mostly vertical arrangement. Another embodiment of the cooling device locates one or more cartridges 310 at the upper back of the wearer near where the two side straps 346 connect. In such embodiment, the gas distribution channel 316 runs along the top of the side straps 346 as well as the top of the cups 342 and each cartridge 310 is in contact with or contained by a frame 320.

Although FIG. 3 shows the gas cartridge 310 in an upright configuration, it should be understood that one embodiment incorporated in a bra uses the opposite cartridge 210 orientation illustrated in FIG. 2b along with the gas manifold design to facilitate heat exchange

The foregoing is merely illustrative of the principles of this invention, and various modifications can be made by those skilled in the art without departing from the scope and spirit of the invention. It should be understood, for example, that the various components, materials, and designs of the embodiments described herein can be mixed and matched in various ways in order to achieve the overall goal of the invention to effectively cool down the body of the wearer by using cool vented gas and the cartridge as a heat sink. The basic components of the various embodiments of the cooling device described herein can be used in any number of applications and in any number of configurations consistent with the general principles of the invention.

While this invention has been particularly shown and described with reference to preferred embodiments, it will be understood by those skilled in the art that various changes in form and detail may be made therein without departing from the spirit and scope of the invention. The inventor expects skilled artisans to employ such variations as appropriate, and the inventor intends the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A portable body cooling device comprising:
a compressed gas container;
a valve connected to the compressed gas container;
at least one gas distribution channel in communication with the compressed gas container by way of the valve, wherein said at least one gas distribution channel comprises at least one gas discharge port such that when the valve is activated gas travels out of the container, through the valve, along the distribution channel and exits out of a discharge port.

2. The portable body cooling device of claim 1 further comprising:
a conductive material in contact with the gas container.

3. The portable body cooling device of claim 1 wherein the at least one gas distribution channel comprises a conductive material.

4. The portable body cooling device of claim 1 wherein such device comprises a pendant housing the gas container.

5. The portable body cooling device of claim 1 wherein such device attaches to an article of clothing.

6. The portable body cooling device of claim 1 wherein such device is built into a woman's bra.

7. The portable body cooling device of claim 1 wherein the gas distribution channel comprises a necklace.

8. The portable body cooling device of claim 1 further comprising a gas manifold in between and in communication with the valve and at least one gas distribution channel.

9. The portable body cooling device of claim 1 wherein the gas container consists of a CO2 cartridge.

10. The portable body cooling device of claim 1 wherein the gas container is in contact with a frame, wherein said frame comprises a heat conducting material on one side and an insulative material on the other side.

11. The portable body cooling device of claim 10 wherein the frame comprises a gas manifold through which gas released from the gas container travels prior to entering the at least one gas distribution channel.

12. A personal cooling device comprising:
a CO2 cartridge mounted in a frame, wherein said frame comprises heat conductive material on a first side and insulative material on a second side, wherein said cartridge is connected to a valve, wherein further said valve is in gas communication with a gas manifold, wherein further said gas manifold is in gas communication with at least one gas distribution channel, wherein further said gas distribution channel comprises a plurality of gas ports.

13. The cooling device of claim 12 wherein the frame is incorporated in a bra.

14. The cooling device of claim 12 wherein the frame is incorporated in a pendant.

15. The cooling device of claim 14 wherein the cooling device further comprises a gas distribution channel in gas communication with the gas manifold and acting as support for said pendant when worn.

16. The cooling device of claim 12 wherein the frame is shaped to fit the contours of a wearer's breasts.

17. The personal cooling device of claim 12 wherein the cartridge is encased in a gas manifold, wherein further said gas manifold is in gas communication with the valve and plurality of gas parts.

18. A method for cooling the skin of the wearer of a portable cooling device, wherein said cooling device comprises a gas cartridge in gas communication with at least one gas distribution channel, said gas distribution channel having a plurality of gas ports, and further wherein said gas cartridge is in contact with a heat conductive frame, the method comprising the steps of
a) placing the frame in contact with a user's skin,
b) releasing gas from the cartridge into the gas distribution channel,
c) venting the released gas through gas ports onto the skin of the wearer.
